(19) United States
(12) Patent Application Publication
(10) Pub. No.: US 2006/0147968 A1
(43) Pub. Date: Jul. 6, 2006

(54) Biochip substrate used for immobilizing biomaterials

(75) Inventors: I-Cherng Chen, Hsinchu Hsien (TW); Cheng-Liang Hsu, Hsinchu (TW); Chung-I Kuo, Hsinchu (TW); Jyisy Yang, Hsinchu (TW); Ruo-Lan Joan Chang, Hsinchu (TW)

Correspondence Address: BIRCH STEWART KOLASCH & BIRCH, PO BOX 747, FALLS CHURCH, VA, 22040-0747, US

(73) Assignee: Industrial Technology Research Institute

(21) Appl. No.: 11/316,968

(22) Filed: Dec. 27, 2005

(30) Foreign Application Priority Data
Dec. 31, 2004 (TW) 093141860
Dec. 19, 2005 (TW) 094145007

(51) Int. Cl. C12Q 1/68; C12M 1/34

(52) U.S. Cl. 435/6; 435/287.2; 977/924

(57) ABSTRACT

The present invention provides a biochip substrate used for immobilizing biomaterials, which has a matrix and at least one metal oxide thread-like nano-object formed vertically on a surface of the matrix. The metal oxide thread-like nano-object is employed for immobilizing the biomaterial onto the matrix of the chip substrate directly, without complicated immobilizing processes by using cross-linking agents or curing agents, and thereby simplifying the process of fabrication. Arrangement as such makes it suitable for purification of a biomaterial. On the other hand, the metal oxide thread-like nano-object characterized by having an enormous surface area is suitable for fabricating immunosensor chip and capable of maintaining high activity and reactivity of biomaterials, so as to increase speed and yield of reactions.

### FIELD OF THE INVENTION

The present invention relates to biochip substrates, and more particularly, to a biochip substrate used for immobilizing biomaterials.

### BACKGROUND OF THE INVENTION

Recently, biochips have been widely used in many technical fields like gene expression, cancer classification, new drug development, disease detection, food technology, agricultural development, and the like. Apparently, biochip technology has become a very important and powerful analysis tool for the future.

Further, following by boosting development of nano-technology and micro-electro-mechanical technology, laborious and time-consuming detection technique such as identifying gene function one by one has been discarded. Contributed by the latest analysis technology, even the gigantic database retrieved from current human genome can now be analyzed at a glance by a finger-size biochip.

However, as a gene function is expressed through activities of a protein that is transcribed from the gene, analyzing protein function directly is thus more meaningful and indicative. Further, as protein expresses its activity by different structures that is very sensitive and may be affected by slightly changes in the surrounding environment, to immobilize the protein onto the chip without damaging or affecting the function or property of the protein has become a great challenge in biochip technology.

Nowadays, biochips provided in the market still require a complicated fabrication method to immobilize proteins onto the chips. For instance, as described in PCT/SE2003/001473 patent application, a chemical agent comprising biological molecules and labeling sequence is employed to immobilize the detected biological molecules (such as proteins and the like) onto the matrix; and as described in U.S. application Ser. No. 10/302,456, a platform with a connecting member is employed for connecting to the matrix, and then biotins are employed to form biochemical bonding with peptides.

Nevertheless, the function or property of the protein may be affected or damaged by any chemical agent employed in the fabrication process. Furthermore, any immobilizing process employing any one of the followings such as covalent bonding, ionic bonding, imbedding or cross-linking may overreact the matrix that is to be immobilized, damage the active structure or function of the biomaterial, or make the matrix unable to form a firm bonding with the biomaterial.

For instance, today's immuno-biochemical sensor chip requires the use of a cross-linking agent or a curing agent to immobilize the proteins onto the sensor chip, and the chip must be treated with a chemical agent first in order to form the functional groups for proceeding chemical reaction. In this case, if a quartz plate is going to be a matrix for further use, it must be treated by acid or alkali first so as to form hydroxy groups first, and then coupling with proteins via a cross-linking agent. Accordingly, the foregoing fabrication process is extremely complicated and laborious and causes an overreaction between the proteins and a crossing-linking agent, wherein the biochemical activity of the proteins is degraded. This thereby makes the quality of controlled of this kind of biochemical sensor chip uneasy to be controlled.

In short, conventional immobilizing technique is time-consuming and laborious, and may degrade the structure and changes function of biomaterial such as proteins; moreover, because the biomaterial is unstable, the bonding between proteins with the matrix will thus be ionized and separated, thereby affecting the experiment result badly.

Conventional technique as such is really not applicable for the industry.

Due to the drawbacks of the conventional technique having complicated structures, higher cost of production, the inconvenient and labor-consuming process of disassembling, requiring a larger space for storage, even more, and affecting the structure of the main machine. Therefore, what would be desirable is to develop a socket-filling member for an electronic device so as to simplify the structure, to reduce the cost of production, to simplify the process of disassembling and to reduce the storage space, so that the internal of the machine can be operated well.

### SUMMARY OF THE INVENTION

In light of the above prior-art drawbacks, a primary objective of the present invention is to provide a biochip substrate that does not require the use of a cross-linking agent or a curing agent.

Another objective of the present invention is to provide a biochip substrate of an immuno-biochip that can be fabricated easily.

Still another objective of the present invention is to provide a biochip substrate that is employed to fabricate an immuno-biochip with high activity and reactivity.

A further objective of the present invention is to provide a method of fabricating a biochip substrate for purifying biomaterials.

In accordance with the foregoing and other objectives, the present invention proposes a biochip substrate used for immobilizing biomaterials, comprising a matrix and at least one metal oxide threadlike nano-object vertically formed on a surface of the matrix. The present invention employs the metal oxide threadlike nano-object formed vertically on the surface of the matrix to immobilize the biomaterial onto the matrix of the chip substrate directly, without employing complicated immobilizing processes requiring the use of cross-linking agents or curing agents. Arrangement as such has the advantage of providing a much simpler fabrication process for manufacturing biochip substrates, and is also capable of being applicable to purify biomaterials. Furthermore, the metal oxide threadlike nano-object characterized by having an enormous surface area is suitable for fabricating immunosensor chip and capable of maintaining high activity and reactivity of biomaterials, so as to increase speed and yield of reactions.

### BRIEF DESCRIPTION OF THE DRAWINGS

The present invention can be more fully understood by reading the following detailed description of the preferred embodiments, with reference made to the accompanying drawings, wherein:

FIG. 1 is a cross-sectional view of a structure of a biochip substrate manufactured according to a preferred embodiment of the present invention;

FIG. 2 is a Scanning Electron Microscopy (SEM) image showing a needle-like structure of each metal oxide thread-like object of a biochip substrate according to a preferred embodiment of the present invention;

FIG. 3 is a Scanning Electron Microscopy (SEM) image showing a wart-like structure of each metal oxide thread-like object of a biochip substrate according to a preferred embodiment of the present invention;

FIG. 4 is a Scanning Electron Microscopy (SEM) image showing a double-layered pillar-like structure of each metal oxide thread-like object of a biochip substrate according to a preferred embodiment of the present invention;

FIG. 5 is a Scanning Electron Microscopy (SEM) image showing a bar-like structure of each metal oxide thread-like object of a biochip substrate according to a preferred embodiment of the present invention;

FIG. 6 is a flow chat showing a fabrication method of a biochip and a detection method using thereof;

FIG. 7 is a Scanning Electron Microscopy (SEM) image showing a structure of a biochip substrate manufactured in accordance with Experiment 1 of the present invention;

FIG. 8 is a chart showing an infrared spectrum of a biochip fabricated in accordance with Experiment 2 of the present invention;

FIG. 9 a chart showing an infrared spectrum of a biochip fabricated in accordance with Experiment 3 of the present invention;

FIG. 10 a graph showing a result of a hydrostability test of a biochip having a wave number at 1572 cm⁻¹ and fabricated in accordance with Experiment 3 of the present invention;

FIG. 11 is a chart showing an infrared spectrum of a biochip fabricated in accordance with Experiment 4 of the present invention;

FIG. 12 a graph showing a result of a hydrostability test of a biochip having a wave number at 1572 cm⁻¹ and fabricated in accordance with Experiment 4 of the present invention;

FIG. 13 is a chart showing an infrared spectrum of a biochip fabricated in accordance with Experiment 5 of the present invention;

FIG. 14 a graph showing a result of a hydrostability test of a biochip having a wave number at 1572 cm⁻¹ and fabricated in accordance with Experiment 3 of the present invention;

FIG. 15 is a chart showing an infrared spectrum of a biochip fabricated in accordance with Experiment 6 of the present invention; and

FIG. 16 is a chart showing an infrared spectrum of a biochip fabricated in accordance with Experiment 7 of the present invention.

### DETAILED DESCRIPTION OF THE PREFERRED EMBODIMENTS

The following embodiments are used to describe the present invention, but they are not restrictive in all aspects.

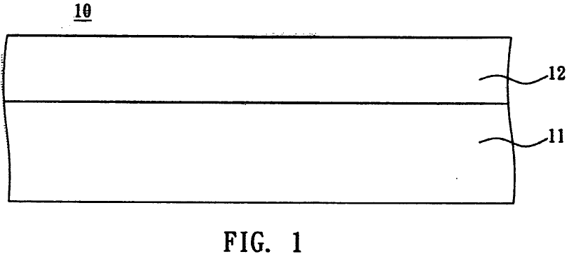

As shown in FIG. 1, a biochip substrate of the present invention mainly comprises a matrix 11 and at least one metal oxide thread-like nano-object 12 formed vertically on a surface of the matrix 11, wherein the metal oxide thread-like nano-object 12 has a sharp end for immobilizing biomaterials. A variety of materials may be selected for fabricating the matrix of the biochip, and it is depended upon the purpose of product. For instance, the material of the matrix may be selected from, but not limited to, at least one of a group consisting of silicon, glass, metal foil, metal complex, and transparent conductive film and the like.

The present invention employs the metal oxide thread-like nano-object formed vertically on the surface of the matrix of the biochip substrate to immobilize the biomaterial. As used herein, the term “thread-like nano-object” refers to a thread-like structure having a diameter that is within 1˜1300 nm. Further, the metal oxide thread-like nano-object of the present invention for immobilizing biomaterial is preferably formed in a width/height ratio of 1:1 to 1:100, and more preferably formed in a width/height ration of 1:10 to 1:100.

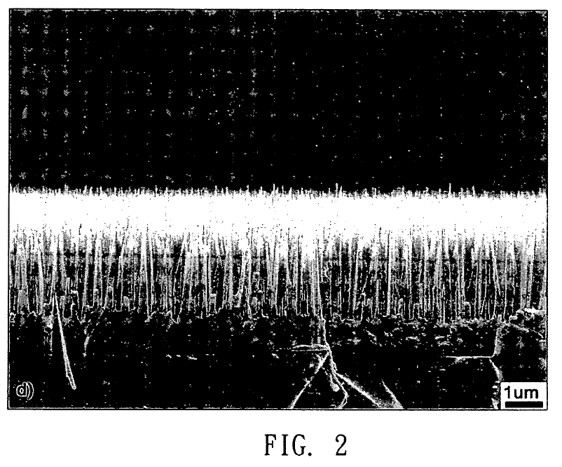

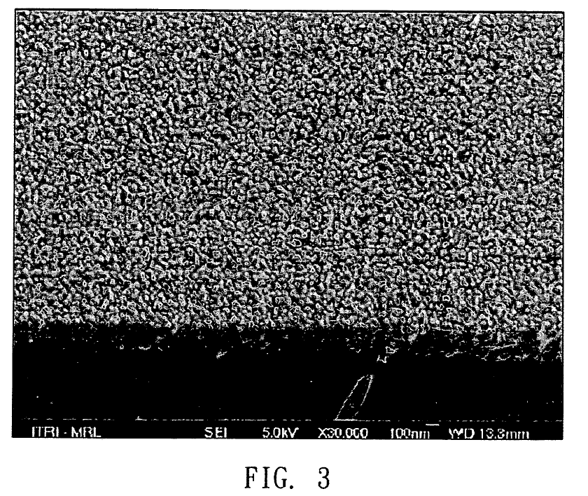

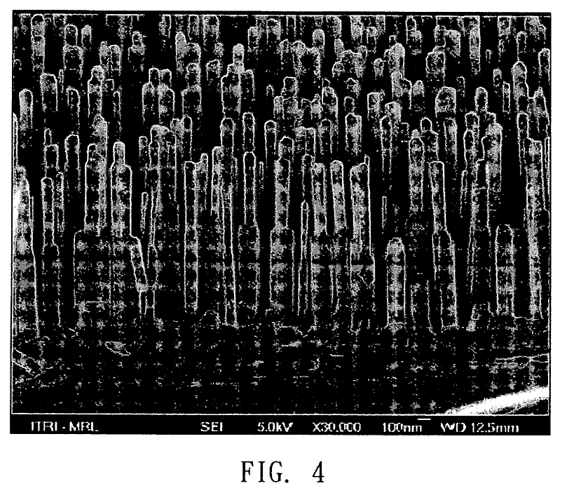

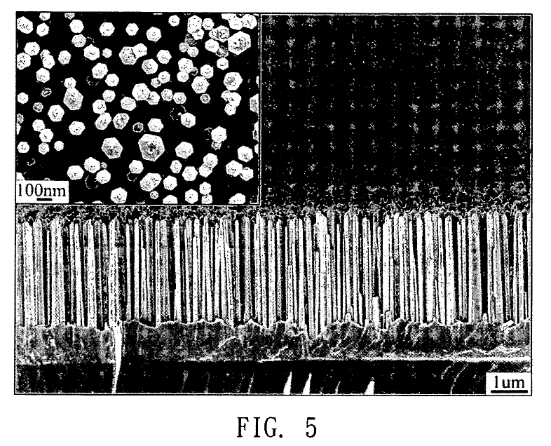

The length of the metal oxide thread-like nano-object is preferably formed within a range of from about 0.1 μm to about 5 μm, and more preferably formed within a range of from about 2.5 μm to about 5 μm. Further, the metal oxide thread-like nano-object is preferably formed at a temperature that is above 600° C. and is more preferably formed above 650° C. Furthermore, the metal oxide thread-like nano-object is formed with a sharp end for immobilizing the biomaterial, wherein the sharp end may be formed in a linear-like shape, a particle-like shape, a needle-like shape (as shown in FIG. 2), a wart-like shape (as shown in FIG. 3), a double-layered pillar-like shape (as shown in FIG. 4), a bar-like shape (as shown in FIG. 5), a rhombus-like shape, a plate-like shape, a horn-like shape, and a chunck-like shape etc, so as to immobilize the biomaterials such as proteins onto the biochip substrate without using any other cross-linking agent or curing agent. The metal oxide thread-like nano-object formed vertically on the surface of the matrix is not arranged in any particular shape. For example, it may be arranged into a rectangle shape, a round shape, a continuous-linear shape, a scattered spots shape or an irregular shape.

The metal oxide thread-like nano-object of the biochip substrate of the present invention may be used for immobilizing any biomaterial. For instance, the biomaterials may be selected from, but not limited to, at least one of a group consisting of peptide, enzyme, antigen, antibody, protein, virus, protein particle, lysosome, hormone, and the like. As the biochip substrate of the present invention does not require any complicated immobilizing process using any cross-linking agent or curing agent, thereby the activities or functions of the biomaterials would not be degraded or impaired. The biochip substrate disclosed in the present invention is applicable to fabrications of protein chip, biochemical reaction chip, biosensor, detection chip, immunosensor chip, and drug screening chip, and the like. The biochip substrate is also applicable to a Lab-on-a-Chip system, a protein purification device, a biomaterial screening and separating system, and the like.

The biochip substrate disclosed in the present invention may be employed for purifying biomaterial. The method for purifying biomaterial comprises steps as follows: (a) vertically forming at least one metal oxide thread-like nano-object on a surface of a matrix so as to fabricate a chip substrate, wherein the metal oxide thread-like nano-object comprises a bottom surface for coupling to the surface of the matrix and a top surface for immobilizing at least one biomaterial; and (b) coupling the biomaterial with the metal oxide thread-like nano-object of the chip substrate so as to immobilize the biomaterials onto the surface of the metal oxide thread-like nano-object.

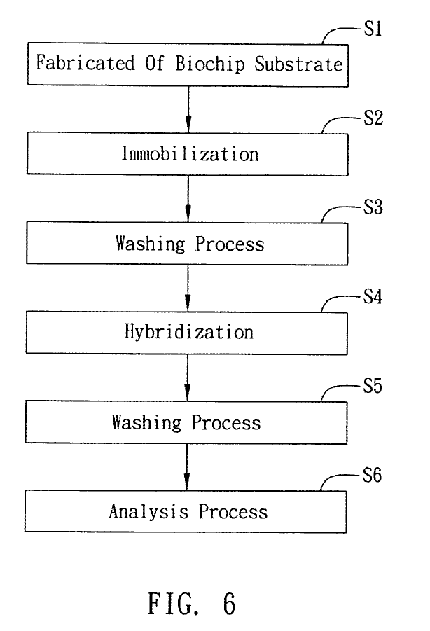

FIG. 6 is a flow chat showing a fabrication method of a biochip and a detection method using thereof.

In step S1, a biochip substrate is fabricated, wherein a metal oxide thread-like nano-object is formed on a matrix thereof. Furthermore, the metal oxide thread-like nano-object may be zinc oxide thread-like nano-object or other metal oxide nanomaterial.

In step S2, an immobilization process is performed, wherein proteins such as antigens or antibodies are placed on the biochip substrate. The reaction progress of immobilization may be proceeded under an appropriate condition, for instance, the reaction may be proceeded at a temperature around 4° C. for 16 hours.

In step S3, a washing step is performed, wherein the biochip is washed by a phosphate buffer saline (PBS) on a shaker for 10 minutes, and then a fresh phosphate buffer saline (PBS) is applied thereto. The washing step is further repeated twice so as to complete the fabrication process of the biochip.

In step S4, a hybridization process is performed involving diluting the antigens or antibodies by the phosphate buffer saline and having the antigens or antibodies reacted with the fabricated biochip to proceed a detection process.

In step S5, the washing step is performed again by using the phosphate buffer saline to proceed the washing step.

In step S6, a measuring process is performed. For instance, an infrared spectrometer or an infrared micro-spectrometer may be employed to measure the spectrum of light that is transmitted, reflected or absorbed. A conventional fluorescent reaction or a fluorescent secondary detection protein reaction may also be used as a measuring method to do the detection process.

The present invention is characterized by using the metal oxide thread-like nano-object to immobilize the biomaterial onto the biochip substrate directly, so that the biomaterials such as proteins may be immobilized to the biochip substrate directly so as to reduce the possibility of protein denaturalization or function impairment. On the other hand, the metal oxide thread-like nano-object is characterized by having an enormous surface area, which is capable of maintaining high activity and reactivity of biomaterials and increasing speed and yield of reactions. Accordingly, the present invention provides a great advantage of improving the benefit of related industry in purifying biomaterials.

### EXAMPLES

### Experiment 1—Formation of a Biochip Substrate

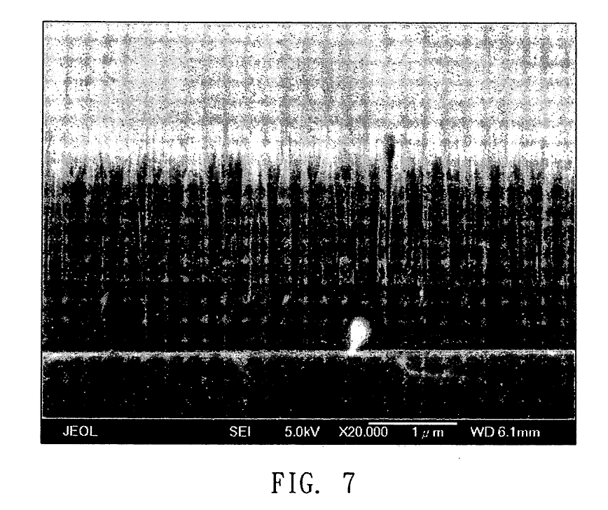

The formation of a biochip substrate comprises the following steps: forming zinc oxide thread-like nano-objects array on a surface of a silicon matrix; washing with water for 30 minutes; and then air-drying at room temperature. The structure of the biochip substrate is shown by a scanning electronic microscopy (SEM) image, as shown in FIG. 7.

### Experiment 2—Formation of a Biochip

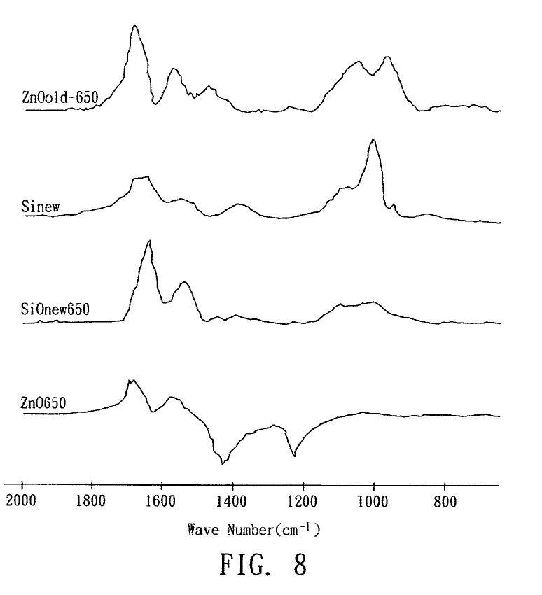

In accordance with the procedures disclosed in Experiment 1, four kinds of biochip substrates (a sample of silicon substrate formed with ZnO-1pillar-like zinc oxide thread-like nano-objects; a sample of Si/SiO2 substrate formed with ZnO-2 pillar-like zinc oxide thread-like nano-objects; a sample of glass substrate formed with ZnO-3 long bar-like zinc oxide thread-like nano-objects; a sample of glass substrate formed with ZnO-4 short bar-like zinc oxide thread-like nano-objects) are fabricated, comprising steps as follows: preparing an antigen solution by adding 5% v/v horse immunoglobulin (horse Ig-G) into a phosphate buffer saline (PBS) solution; placing 5 μl of antigens on the biochip substrate; and then air-drying at room temperature. An infrared spectrometer is employed to proceed an analysis process, wherein the absorption spectrum of —C═O is observed at a wave number of 1680 cm⁻¹, the absorption spectrum of —NH₂ is observed at a wave number of 1572 cm⁻¹, and the absorption spectra of the interaction of C—O therebetween and the interaction of C—N—H therebetween is respectively observed at a wave number of 1120 cm⁻¹, as shown in FIG. 8.

### Experiment 3—a Hydrostability Test for the Biochip

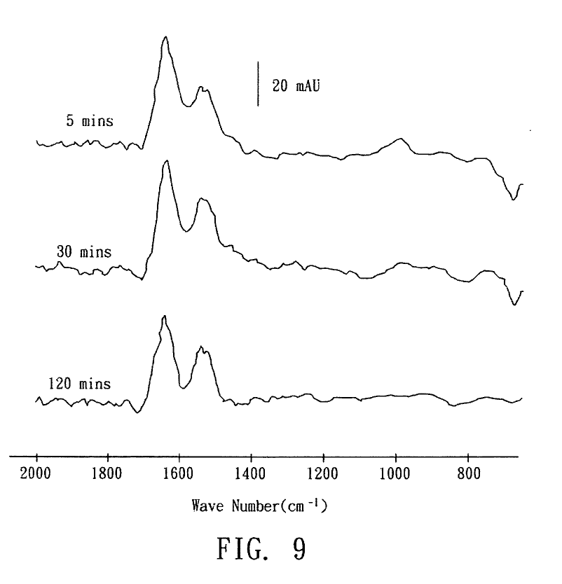

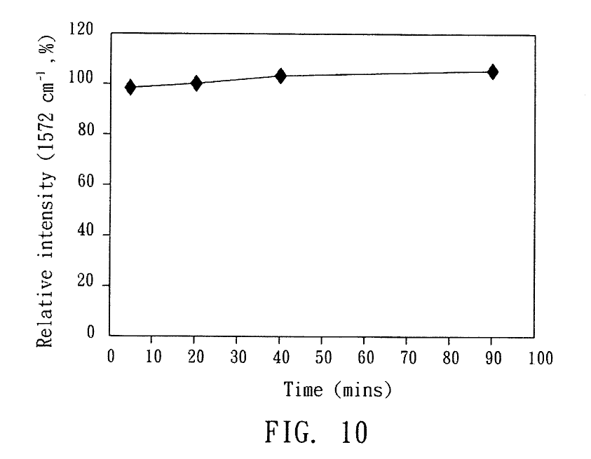

In accordance with the procedures disclosed in Experiment 1, a biochip substrates (0225B, a sample of chip substrate having zinc oxide thread-like nano-object formed in a needle-like shape) is fabricated, comprising steps as follows: preparing an antigen solution by adding 5% v/v horse immunoglobulin (horse Ig-G) into a phosphate buffer saline (PBS) solution; placing 5 μl of antigens on the biochip substrate; air-drying at room temperature; washing with water; after respectively washing for 5 minutes, 30 minutes, and 120 minutes, employing an infrared spectrometer to proceed an analysis process, wherein the results thereof are shown in FIGS. 9 and 10.

### Experiment 4—a Hydrostability Test for the Biochip

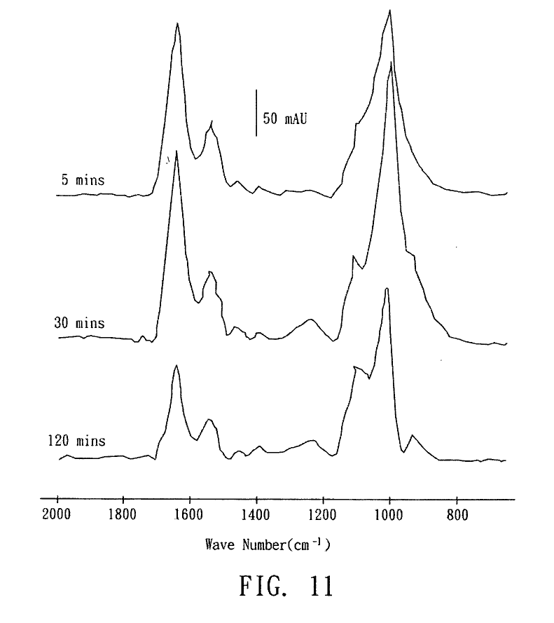

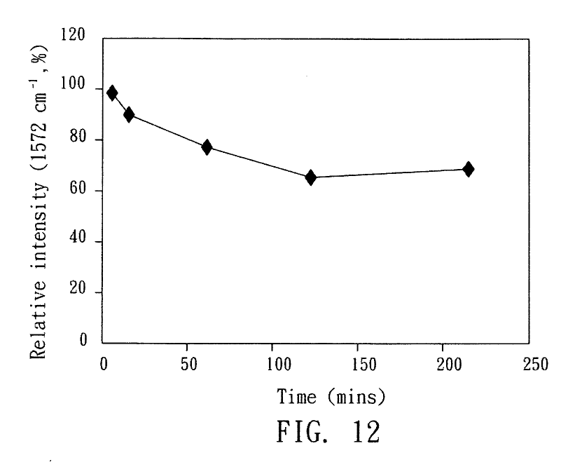

In accordance with the procedures disclosed in Experiment 1, a chip substrates (f194, a sample of silicon chip substrate having zinc oxide thread-like nano-object formed in a pillar-like shape) is fabricated, comprising steps as follows: preparing antigens by adding 5% v/v horse immunoglobulin (horse Ig-G) into a phosphate buffer saline (PBS) solution; placing 5 μl of antigens on the biochip substrate; air-drying at room temperature; washing with water; after respectively washing for 5 minutes, 30 minutes, and 120 minutes, employing an infrared spectrometer to proceed an analysis process, wherein the results thereof are shown in FIGS. 11 and 12.

### Experiment 5—a Hydrostability Test for the Biochip

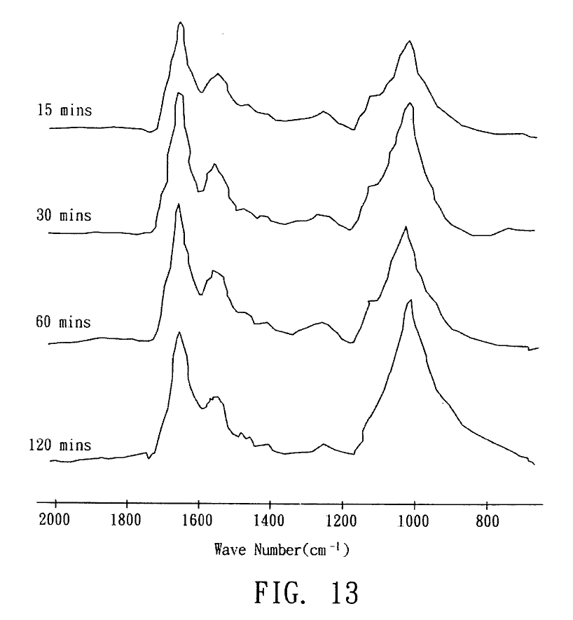

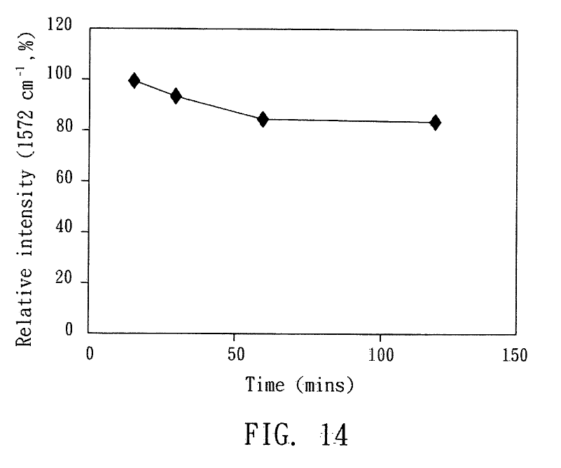

In accordance with the procedures disclosed in Experiment 1, a biochip substrates (SiOnew-650, a sample of silicon chip substrate having zinc oxide thread-like nano-object formed in a long bar-like shape) is fabricated, comprising steps as follows: preparing an antigen solution by adding 5% v/v horse immunoglobulin (horse Ig-G) into a phosphate buffer saline (PBS) solution; placing 5 μl of antigens on the biochip substrate; air-drying at room temperature; washing with water; after respectively washing for 15 minutes, 30 minutes, 60 minutes, and 120 minutes, employing an infrared spectrometer to proceed an analysis process, wherein the results thereof are shown in FIGS. 13 and 14.

### Experiment 6—an Assay for Detection of Biochip Activity

In accordance with the procedures disclosed in Experiment 1, a biochip substrates (0225B, a sample of silicon chip substrate having zinc oxide thread-like nano-object formed in a needle-like shape) is fabricated, comprising steps as follows: preparing an antigen solution by adding 5% v/v horse immunoglobulin (horse Ig-G) into a phosphate buffer saline (PBS) solution; placing 5 μl of antigens on the biochip substrate; and air-drying at room temperature.

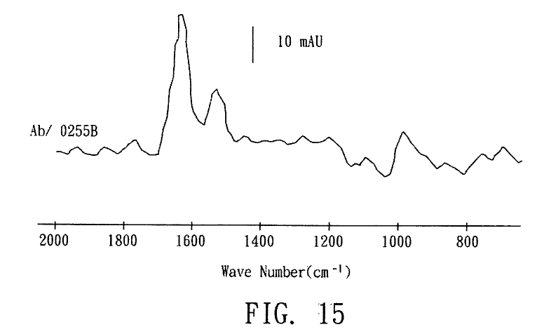

An corresponding antibody solution (anti-horse IgG) is prepared at a buffering solution/antibody volume ratio of 1000:1. The chip is washed with a phosphate buffer saline solution (pH 7.4) for 30 minutes. Then the chip is placed into a 10 ml antibody solution and waited for a period, so as to allow the reaction to progress for 2 hours at room temperature. Last, an infrared spectrometer is employed to do an analysis process, wherein the result thereof is shown in FIG. 15.

### Experiment 7—an Assay for Detection of Biochip Activity

In accordance with the procedures disclosed in Experiment 1, a chip substrates (ZnO650, a sample of Si/SiO2 chip substrate having zinc oxide thread-like nano-object formed in a pillar-like shape) is fabricated, comprising steps as follows: preparing an antigen solution by adding 5% v/v horse immunoglobulin (horse Ig-G) into a phosphate buffer saline (PBS) solution; placing 5 μl of antigens on the biochip substrate; and air-drying at room temperature.

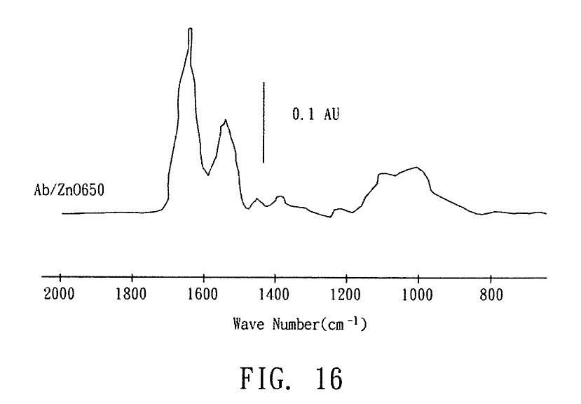

An corresponding antibody solution (anti-horse IgG) is prepared at a buffering solution/antibody volume ratio of 1000:1. The chip is washed with a phosphate buffer saline solution (pH 7.4) for 30 minutes. Then the chip is placed into a 10 ml antibody solution and waited for a period, so as to allow the reaction to progress for 2 hours at room temperature. Last, an infrared spectrometer is employed to do an analysis process, wherein the result thereof is shown in FIG. 16.

The invention has been described using exemplary preferred embodiments. However, it is to be understood that the scope of the invention is not limited to the disclosed embodiments. On the contrary, it is intended to cover various modifications and similar arrangement. The scope of the claims therefore should be accorded the broadest interpretation so as to encompass all such modifications and similar arrangements.

What is claimed is:

1. A biochip substrate used for immobilizing biomaterials comprising:
a matrix; and
at least one metal oxide thread-like nano-object formed vertically on a surface of the matrix.

2. The biochip substrate of claim 1, wherein the metal oxide thread-like nano-object is formed in a width/height ratio of 1:1 to 1:100.

3. The biochip substrate of claim 1, wherein the metal oxide thread-like nano-object is formed in a width/height ratio of 1:10 to 1:100.

4. The biochip substrate of claim 1, wherein the length of the metal oxide thread-like nano-object is formed within a range of from about 0.1 μm to about 5 μm.

5. The biochip substrate of claim 4, wherein the length of the metal oxide thread-like nano-object is formed within a range of from about 2.5 μm to about 5 μm.

6. The biochip substrate of claim 5, wherein the metal oxide thread-like nano-object is formed with a sharp end.

7. The biochip substrate of claim 6, wherein the metal oxide thread-like nano-object with a sharp end is formed in at least one of a linear-like shape, a particle-like shape, a needle-like shape, a wart-like shape, a double-layered pillar-like shape, a bar-like shape, a rhombus-like shape, a plate-like shape, a horn-like shape, and a chunck-like shape.

8. The biochip substrate of claim 1, wherein the metal oxide thread-like nano-object formed vertically on the surface of the matrix is arranged into at least one of a rectangle shape, a round shape, a continuous-linear shape, a scattered spots shape and an irregular shape.

9. The biochip substrate of claim 1, wherein the metal oxide thread-like nano-object is formed at temperature above 600° C.

10. The biochip substrate of claim 1, wherein the metal oxide substance is zinc oxide.

11. The biochip substrate of claim 1, wherein the matrix is selected from at least one of a group consisting of silicon, glass, metal foil, metal complex, and transparent conductive film.
